(19) United States
(12) Patent Application Publication
(10) Pub. No.: US 2012/0100564 A1
(43) Pub. Date: Apr. 26, 2012

(54) Prediction of Memapsin 2 Cleavage Sites

(76) Inventors: Jordan Tang, Edmond, OK (US); Xiaoman Li, Oklahoma City, OK (US); Jean Hartsuck, Oklahoma City, OK (US)

(21) Appl. No.: 13/221,467

(22) Filed: Aug. 30, 2011

Related U.S. Application Data
(60) Provisional application No. 61/379,604, filed on Sep. 2, 2010.

(51) Int. Cl. C12Q 1/37; G06F 17/10

(52) U.S. Cl. 435/23; 703/2

(57) ABSTRACT

Aspartic proteases such as mempasin-2 are import enzymes, playing roles in a variety of diseases. The inventors have developed a model to predict the cleavage sites and preferences for memapsin 2 substrates.

The present application claims benefit of priority to U.S. Provisional Application Ser. No. 61/379,604, filed Sep. 2, 2010, the entire contents of which are hereby incorporated by reference.

The United States Government owns rights in the invention pursuant to funding from the National Institutes of Health under Grant No. AG-18933.

### BACKGROUND OF THE INVENTION

I. Field of the Invention

The present invention relates generally to the fields of enzymology and biochemistry. More particularly, it concerns the prediction of cleavage sites in known and unknown substrates for the memapsin 2 protease.

II. Description of Related Art

Alzheimer's disease (AD) is the most common form of dementia among older people. Scientists believe that up to 4 million Americans suffer from AD. The disease usually begins after age 60, and risk goes up with age. While younger people also may get AD, it is much less common. About 3 percent of men and women ages 65 to 74 have AD, and nearly half of those age 85 and older may have the disease. While the subject of intensive research, the precise causes of AD are still unknown, and there is no cure.

AD attacks parts of the brain that control thought, memory and language. It was identified in 1906 by German doctor Dr. Alois Alzheimer who noticed changes in the brain tissue of a woman who had died of an unusual mental illness. He found abnormal clumps, now called amyloid “plaques,” and tangled bundles of fibers, now called neurofibrillary “tangles.” Today, these plaques and tangles in the brain are considered hallmarks of AD.

The production, aggregation, and accumulation of amyloid β-protein (Aβ), the major constituent of the amyloid plaque, in the brain are initial steps in the pathogenesis of AD. Aβ is generated by the intracellular processing of amyloid β precursor protein (APP, see FIG. 1) (Selkoe, 2001), a type I membrane protein (Kang et al., 1987), by proteases β-secretase (memapsin 2 or BACE1) and γ-secretase. Thus, memapsin 2 constitutes an important potential target for AD therapies, and understanding its activity and target specificity is critical to designing antagonists of this enzyme.

### SUMMARY OF THE INVENTION

Thus, in accordance with the present invention, there is provided a method of predicting a relative memapsin 2 cleavage efficiency for a site in a peptide or polypeptide sequence comprising (a) providing a site comprising an amino acid sequence of at least five residues in length, wherein consecutive residues of said sequence are assigned as subsites P₃, P₂, P₁, P₁′, and P₂′ in an N- to C-terminal order, wherein cleavage occurs between P₁ and P₁′; and (b) obtaining a cleavage preference value for each of subsites P₃, P₂, P₁, P₁′, and P₂′ based on the following formula:

Q=Exp(Σwᵢ ln aᵢ)

wherein Q is value for memapsin 2 cleavage efficiency, aᵢ is the relative k_{cat}/K_{M} value for Pᵢ from the following chart:

| | |
|---|---|
| Upstream | Downstream |

| | P₄ | P₃ | P₂ | P₁ | P_{1′} | P_{2′} |
|---|---|---|---|---|---|---|
| W | 0.19 | 0.01* | 0.01 | 0.01* | 0.03 | 0.02 |
| F | 0.17 | 0.17 | 0.69 | 0.88 | 0.14 | 0.92 |
| Y | 0.05 | 0.02 | 0.58 | 0.29 | 0.37 | 0.61 |
| M | 0.45 | 0.36 | 0.97 | 0.54 | 1.47 | 0.73 |
| L | 0.25 | 1.23 | 0.59 | 1.00 | 0.30 | 0.94 |
| I | 0.11 | 1.37 | 0.01* | 0.01* | 0.13 | 1.38 |
| V | 0.17 | 1.00 | 0.01* | 0.01* | 0.20 | 1.41 |
| A | 0.12 | 0.39 | 0.34 | 0.02 | 1.00 | 1.00 |
| G | 0.39 | 0.02 | 0.02 | 0.04 | 0.04 | 0.16 |
| T | 0.24 | 0.38 | 0.01* | 0.16 | 0.24 | 0.87 |
| S | 0.14 | 0.22 | 0.50 | 0.07 | 0.67 | 0.48 |
| Q | 0.85 | 0.05 | 0.17 | 0.01* | 1.09 | 0.13 |
| N | 0.43 | 0.01* | 1.00 | 0.02 | 0.04 | 0.03 |
| E | 1.00 | 0.63 | 0.53 | 0.01* | 1.32 | 0.96 |
| D | 0.64 | 0.11 | 1.22 | 0.06 | 0.82 | 0.02 |
| H | 0.29 | 0.53 | 0.01* | 0.02 | 0.01* | 0.01* |
| R | 0.24 | 0.01* | 0.01* | 0.01* | 0.06 | 0.01* |
| K | 0.01* | 0.29 | 0.10 | 0.01* | 0.06 | 0.02 |
| P | 0.25 | 0.37 | 0.01* | 0.01* | 0.01* | 0.01* |

and wᵢ is the weighing factor of each Pᵢ as shown below:

| W4 | W3 | W2 | W1 | W1′ | W2′ |
|---|---|---|---|---|---|
| 0.89 | 3.50 | 1.02 | 6.26 | 0.38 | 1.09 |

The method may further comprise assessing a cleavage preference for subsite P₄ based on the preference chart, wherein subsite P₄ is N-terminal to subsite P₃, and creating a predicted k_{cat}/K_{M} for said site based on values from step (b). The peptide or polypeptide may be a known substrate for memapsin 2, or may not be a known substrate for memapsin 2. The peptide or polypeptide may be disease polypeptide. The method may further comprise subjecting said peptide or polypeptide comprising said site to cleavage by memapsin 2, and optionally further comprise determining an actual k_{cat}/K_{M} for the site. The method may further comprising providing said peptide or polypeptide to a subject.

The method may further comprise modifying at least one residue in said site, and optionally performing steps (a) and (b) of claim 1 on the modified site. The method may further comprise providing a site that is modified in at least one residue as compared to the site provided in step (a), and performing step (b) on the modified site, and optionally further comprise preparing a peptide or polypeptide comprising the modified site, and optionally further comprise subjecting the modified peptide or polypeptide comprising said site to cleavage by memapsin 2, and optionally further comprise determining an actual k_{cat}/K_{M} for the modified site. The method may further comprise providing the modified peptide or polypeptide to a subject.

It is contemplated that any method or composition described herein can be implemented with respect to any other method or composition described herein.

Systems and computer readable media are also presented for predicting a relative memapsin 2 cleavage efficiency for a site in a peptide or polypeptide sequence the system. The systems may include computer memory configured to hold information relating to a site comprising an amino acid sequence of at least five residues in length, wherein consecutive residues of said sequence are assigned as subsites P₃, P₂, P₁, P₁′, and P₂′ in an N- to C-terminal order, wherein cleavage occurs between P₁ and P₁′. The systems may also include a computer processor configured to read the information relating to the site from the computer memory and to obtain a cleavage preference value for each of subsites P₃, P₂, P¹, P₁′, and P₂′ based on the following formula:

Q=Exp(Σwᵢ ln aᵢ)

wherein Q is value for memapsin 2 cleavage efficiency, aᵢ is the relative k_{cat}/K_{M} value for Pᵢ and wᵢ is the weighing factor of each Pᵢ as determined by the charts above.

Computer program products are also presented. The computer program products may include a computer readable medium having computer usable program code executable to perform operations for processing data, the operations of the computer program product comprising the steps of the methods described above.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

### BRIEF DESCRIPTION OF THE DRAWINGS

The following drawings form part of the present specification and are included to further demonstrate certain aspects of the present invention.

FIGS. 1A-C: Preference of amino acid residues in the upstream subsites of memapsin 2 substrates. The preference index (see Material and methods) was calculated from the relative initial hydrolytic rates of the mixed substrates and is proportional to the relative I_{cat}/K_{M}. Amino acids (single-letter code) appear in the substrate template sequence at the position designated in each panel (Pₙ). The arrows indicate the residues found in native APP. (FIG. 1A) Complete amino acid residue preference for four subsites (S₅-S₈) derived by competitive hydrolysis assay from peptide mixture P₅ to P₈ and ESI-TOF mass spectrometry. (FIG. 1B) Scheme of determination of subsite specificity by stable-isotope-assisted MALDI-TOF mass spectrometry. (FIG. 1C) Comparison of subsite specificity of upstream subsites, determinate by competitive hydrolysis assay together with stable-isotope-assisted-MALDI-TOF mass spectrometry and ESI mass spectrometry using peptide mixtures containing representative substrates (P₆-1, P₇-1 and P₈-1).

FIG. 2: Correlation of the calculated and observed relative k_{cat}/K_{M} values of different substrates. The calculated relative k_{cat}/K_{M} of different substrates are plotted to the relative k_{cat}/K_{M} of different substrates determined by experiments (the relative k_{cat}/K_{M} of peptide derived from Swedish APP is arbitrarily assigned as 100, the relative k_{cat}/K_{M} of other substrates were determined by normalized to Swedish APP). Logarithmic scale is used for X-, Y-axes. The correlation coefficient for the predicted data to experimental data is 0.97.

FIG. 3: Hydrolysis of cerebellin by memapsin 2. Upper panel: cerebellin only. Lower panel: cerebellin digested with memapsin 2. After digestion, two products appears with the masses of 679.18 Da and 885.24 Da, which are assigned to the fragment GSAKVAF and SAIRSTNH, the N-terminal and C-terminal products generated from the predicted cleavage site respectively.

FIGS. 4A-B: Processing of APP variants by memapsin 2. (FIG. 4A) CAD cell line was transfected with each APP construct followed by Western analysis of cell lysates and conditioned medium. The 5352 antibody was used for detecting full length APP, CTF 99 and CTF 83. sAPPα was detected by Ab 1560. The 22C11 antibody is used for detecting sAPP (sAPPα+sAPPβ). (FIG. 4B) Quantitation of soluble amyloid peptides were performed by ELISA. The -fold increase of different APP mutants compared to APP_{WT} is showed on the top of the bar graph.

### DESCRIPTION OF ILLUSTRATIVE EMBODIMENTS

Aspartic proteases are a family of protease enzymes that use an aspartate residue for catalysis of their peptide substrates. In general, they have two highly-conserved aspartates in the active site and are optimally active at acidic pH. Aspartic proteases are involved in disease such as hypertension, HIV, tumorigenesis, peptic ulcer disease, amyloid disease, malaria and common fungal infections such as candidiasis.

Eukaryotic aspartic proteases include pepsins, cathepsins, and renins. They have a two-domain structure, arising from ancestral duplication. Each domain contributes a catalytic Asp residue, with an extended active site cleft localized between the two lobes of the molecule. One lobe has probably evolved from the other through a gene duplication event in the distant past. In modern-day enzymes, although the three-dimensional structures are very similar, the amino acid sequences are more divergent, except for the catalytic site motif, which is very conserved. The presence and position of disulfide bridges are other conserved features of aspartic peptidases.

As discussed above, memapsin 2, an aspartic protease, is involved in the development of the neurodegenerative disease Alzheimer's Disease (“AD”). Remarkably, though much progress has been made in recent years, there remain relatively few drugs that are useful in the treatment of AD, and almost none that are effective for a high percentage of patients. Thus, there is an urgent need for new and improved drugs and methods of therapy for this condition, as well as inhibitors for other disease-related aspartic proteases. Being able to predict the substrate specificity for aspartic proteases would therefore be of great value in this research.

### I. ALZHEIMER'S DISEASE

AD is a progressive, neurodegenerative disease characterized by memory loss, language deterioration, impaired visuospatial skills, poor judgment, indifferent attitude, but preserved motor function. AD usually begins after age 65, however, its onset may occur as early as age 40, appearing first as memory decline and, over several years, destroying cognition, personality, and ability to function. Confusion and restlessness may also occur. The type, severity, sequence, and progression of mental changes vary widely. The early symptoms of AD, which include forgetfulness and loss of concentration, can be missed easily because they resemble natural signs of aging. Similar symptoms can also result from fatigue, grief, depression, illness, vision or hearing loss, the use of alcohol or certain medications, or simply the burden of too many details to remember at once.

There is no cure for AD and no way to slow the progression of the disease. For some people in the early or middle stages of the disease, medication such as tacrine may alleviate some cognitive symptoms. Aricept (donepezil) and Exelon (rivastigmine) are reversible acetylcholinesterase inhibitors that are indicated for the treatment of mild to moderate dementia of the Alzheimer's type. Also, some medications may help control behavioral symptoms such as sleeplessness, agitation, wandering, anxiety, and depression. These treatments are aimed at making the patient more comfortable.

AD is a progressive disease. The course of the disease varies from person to person. Some people have the disease only for the last 5 years of life, while others may have it for as many as 20 years. The most common cause of death in AD patients is infection.

The molecular aspect of AD is complicated and not yet fully defined. As stated above, AD is characterized by the formation of amyloid plaques and neurofibrillary tangles in the brain, particularly in the hippocampus which is the center for memory processing. Several molecules contribute to these structures: amyloid β protein (Aβ), presenilin (PS), cholesterol, apolipoprotein E (ApoE), and Tau protein. Of these, Aβ appears to play the central role.

Aβ contains approximately 40 amino acid residues. The 42 and 43 residue forms are much more toxic than the 40 residue form. Aβ is generated from an amyloid precursor protein (APP) by sequential proteolysis. One of the enzymes lacks sequence specificity and thus can generate Aβ of varying (39-43) lengths. The toxic forms of Aβ cause abnormal events such as apoptosis, free radical formation, aggregation and inflammation. Presenilin encodes the protease responsible for cleaving APP into Aβ. There are two forms—PS1 and PS2. Mutations in PS1, causing production of Aβ₄₂, are the typical cause of early onset AD.

Cholesterol-reducing agents have been alleged to have AD-preventative capabilities, although no definitive evidence has linked elevated cholesterol to increased risk of AD. However, the discovery that Aβ contains a sphingolipid binding domain lends further credence to this theory. Similarly, ApoE, which is involved in the redistribution of cholesterol, is now believed to contribute to AD development. As discussed above, individuals having the ApoE4 allele, which exhibits the least degree of cholesterol efflux from neurons, are more likely to develop AD.

Tau protein, associated with microtubules in normal brain, forms paired helical filaments (PHFs) in AD-affected brains which are the primary constituent of neurofibrillary tangles. Recent evidence suggests that Aβ proteins may cause hyperphosphorylation of Tau proteins, leading to disassociation from microtubules and aggregation into PHFs.

### II. MEMAPSIN 2

Memapsin 2 (BACE1, β-secretase) is a membrane anchored aspartic protease. Although this enzyme is ubiquitously present in many mammalian organs, its functions in the brain are best studied. One of the most important physiological functions of memapsin 2 is the cleavage of a brain membrane protein β-amyloid precursor protein (APP). The hydrolytic product of APP C-terminal fragment is cleaved again by an intramembrane protease γ-secretase to generate a 40- or 42-residue β-amyloid peptide (Ar). Aβ has been shown to feedback down regulate the synaptic activity in neurons (Kamenetz et al., 2003; Lauren et al., 2009). Also, memapsin 2 produced APP N-terminal fragment is involved in the trimming of neurons and axons in the brain (Nikolaev et al., 2009). However, since excess levels of brain Aβ are intimately related to the pathogenesis of Alzheimer's disease (Selkoe, 1999), there has been intensive effort to develop inhibitor drugs against memapsin 2 (Ghosh et al., 2008). Important to such effort is the detailed knowledge on the specificity preference of this protease. In addition, there has been interest in other possible physiological functions of memapsin 2 that need to be taken into consideration when developing inhibitors. The protease is known to be involved in the processing of neuregulin 1 during neuronal myelination in prenatal mice (Willem et al., 2006; Hu et al., 2006). Other proteins processed by memapsin 2 include the beta-subunit of voltage-gated sodium channels (VGSC βs) (Wong et al., 2005; Kim et al., 2007; Miyazaki et al., 2007), alpha 2,6-sialyltransferase (ST6Gale (Kitazume et al., 2001), P-selectin glycoprotein ligand-1 (PSGL-1) (Lichtenthaler et al., 2003), interleukin-1 Receptor II (IL-IR2) (Kuhn et al., 2007), low density lipoprotein receptor-related protein (LRP) (von Arnim et al., 2005) and amyloid-beta precursor-like proteins (APLPs) (Li and Sudhof, 2004; Pastorino et al., 2004; Walsh et al., 2007). The physiological significance of some of these cleavages has not been clearly delineated and only some of the memapsin 2 cleavage sites on these proteins have been determined. The cleavage of many of these proteins by memapsin 2 was demonstrated in cells over-expressing the substrate protein. Under these conditions, the cleavage of some non-physiological substrates can be enhanced by an increased availability for cleavage or a distorted localization of the over-expressed protein in subcellular compartments. Also, in cellular or in vivo experiments, memapsin 2 cleavage sites may be subjected to additional proteolysis by other cellular proteases thus leading to the erroneous identification of memapsin 2 processing site, such is the case of alpha 2, 6-sialyltransferase (Kitazume et al., 2001; Kitazume et al., 2005). For these reasons, a clear understanding of memapsin 2 specificity with the ability to predict its activity toward different potential cleavage sites would be of assistance to the studies of physiological functions of this protease.

The polypeptide chain of memapsin 2 comprises a N-terminal ecto-catalytic domain, a transmembrane domain and a C-terminal cytosolic domain (Lin et al., 2000). The catalytic domain is homologous to aspartic proteases of the pepsin superfamily in both the amino acid sequence (Lin et al., 2000) and in tertiary structure (Hong et al., 2000). The activity of memapsin 2 is optimal near pH 4 (Ermolieff et al., 2000), as is consistent with its function primarily within endosomal vesicles. The crystal structure of the catalytic domain shows that, like other aspartic proteases, memapsin 2 has a long substrate-binding cleft between the N- and C-terminal lobes that occupies nearly the entire width of the molecule (Hong et al., 2000). The binding positions of transition-state analogues in the protease indicate that the substrate-binding cleft can accommodate 11 to 12 residues, with 7 to 8 residues at the N-terminus side (subsites P₈ to P₁) and 4 at the C-terminal side (subsites P₁′ to P₄′) (Hong et al., 2000; Hong et al., 2002). The inventors reported the residue preferences on 19 amino acids in eight memapsin 2 subsites, from S₄ to S₄′, which are the subsites commonly present in aspartic proteases (Turner et al., 2001). They also reported that memapsin 2 possesses 3 to 4 additional subsites and determined partial residue preferences in three these sites, S₇ S₆ and S₅ (Turner et al., 2004). These data, determined as relative k_{cat}/K_{M}, established that this protease has a somewhat broad specificity in all subsites.

### III. PREDICTIVE VALUES FOR MEMAPSIN 2 CLEAVAGE

The inventors determined the residue preferences on 19 amino acids in 8 memapsin 2 subsites, from S₄ to S₄′, which are the subsites commonly present in aspartic proteases (Turner et aL, 2001). They found that memapsin 2 possesses 3 to 4 additional subsites, and determined partial residue preferences in three these sites, S₇ , S₆ , and S₅ (Turner et al., 2004). These data, determined as relative k_{cat}/K_{M}, established that this protease has a somewhat broad specificity in all subsites. Finally, they determined that the contribution of different subsites to the determination of substrate cleavage sites can be expressed in quantitative terms and further developed a predictive model to determine the probability of cleavage sites in any peptide substrate. Table 5 below presents the substrate preference at each subsite in quantitative terms. At each subsite, the ‘template residue’ was set to have the value of 1.0 and rest of the amino acids are calculated by the ratio of their k_{cal}/K_{M} values to the ‘template residue’. A ‘template residue’ is the residue used in a subsite when it is not being varied to study the specificity. The ‘template residues’ for the eight subsites are, from P4 to P4′, EVNLAAEF (in single letter amino acid code). The values in Table 5 are used in predict memapsin 2 activity on substrates whose sequence determines the residues in subsite positions, thus, obtain the a; values from Table 5, and the wᵢ value from Table 2, for the predictive calculation in the algorithm equation Q=Exp(Σwᵢ ln aᵢ).

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Materials. α-cyan-4 hydroxycinnamic acid, D₀- and D₆-form acetic anhydride , N-hydroxysuccinimide were purchased from Sigma. Peptide (Des-Ser1)-Cerebellin was purchased from Bachem (Bubendorf, Switzerland). All peptides derived from memapsin 2 potential substrates were synthesized by GenScript (Piscataway, N.J.). The ecto-domain of human memapsin 2 was expressed and purified as described previously (Hong et al., 2000). Monoclonal anti-memapsin 2 antibody 3E7 was purchased from Santa Cruz Biotechnology Inc (Santa Cruz, Calif.). Monoclonal anti-APP antibody 1560, MAB348 and polyclonal anti-APP antibody 5352 were purchased from Millipore (Billerica, Mass.). Monoclonal anti-actin antibody was purchased from Abcam (Cambridge, Mass.).

Design of the defined substrate mixtures. Peptide sequence RK (P₁₀)T(P₉)E(P₈)E(P₇)I(P₆)S(P₅)E(P₄)V(P₃)N(P₂)L(P₁)D(P₁′)A(P₂′)E(P₃′)F(P₄′), corresponding to the amino acid sequence of APP with Swedish mutation (APP_{SW}) from P₁₀ to P₄′, was used to be a template to study residue preferences in substrate mixtures (* denotes the cleavage site). An additional arginine (R) was included to facilitate the detection in mass spectrometry. For characterization of upstream subsites outside of subsite P₄, 4 sets of separate substrate mixtures were synthesized by the appropriate cycle of solid-phase peptide synthesis (Synpep, Dublin, Calif.): RKTEEI-[X]-EVNL*DAEF, RKTEE-[X]-SEVNL*DAEF, RKTE-[X]-ISEVNL*DAEF, RKT-[X]-EISEVNL*DAEF (defined as “P₅”, “P₆”, “P₇”, “P₈” respectively, X represents any of the amino acid except cysteine, * denotes the cleavage site). Thus, at each subsite, 19 varied amino acids were accommodated in 4 substrate mixtures, requiring 16 substrate mixtures to characterize those 4 subsites. Each mixture contains an equal-molar of five or six amino acid derivatives differed only by one amino acid at a single subsite. A substrate derived from APPsw, RKTEEISEVNL*DAEF, was also added to each mixture to serve as an internal standard. Three additional sets of mixtures used previously (Turner et al., 2001) were also employed here, RTEE-[X]-SEVNL*AAEF for study of P₆, RTE-[X]-ISEVNL*AAEF for study of P₇, RT-[X]-EISEVNL*AAEF for study of P₈ (defined as “P₆-1”, “P₇-1”, “P₈-1” respectively to distinguish from the peptide mixtures described above, X represents several representing amino acids at that position, * represents the cleavage site).

Kinetic analysis of subsite specificity using ESI-TOF mass spectrometry. Substrate mixtures were dissolved at 10 mg/ml in DMSO and were further diluted to 10 μM in 0.1 M MOPS buffer (pH 4.0). After equilibration at room temperature, the reactions were initiated by the addition of an aliquot of activated memapsin 2 (final concentration is around 60 nM). Aliquots were removed at time intervals, quenched by formic acid. Quantitative analysis was conducted by ESI LC/MS. The system was composed of an Agilent 1100 HPLC, a Clipeus 1×50 mm 5 mm C-18 chromatographic column, and a Bruker MicroTOF ESI-MS (Bruker daltonics, Bremen, Germany). The HPLC buffers used were: A—99.5% H₂O, 0.5% Formic Acid, and B—99.5% Acetonitrile, 0.5% Formic Acid. Separations were conducted using a 5% to 50% B gradient over 4 minutes at a flow rate of 200 μl/min. Ion detection was accomplished using the time of flight instrument in positive reflector mode with ion detection between 200 and 2000 m/z through an ESI interface. Data were analyzed by Quant Analysis software equipped with the ESI mass spectrometer to obtain ion areas of the substrates and their corresponding products in a given reaction. The ratios of individual product to sum of product and its corresponding substrate peptide (relative product formation) from observed ion areas were plotted against time. Relative product formed per unit time was obtained from nonlinear regression analysis of the data representing the initial 15% formation of product using the model:

F=1−e^{-kT}

where F is fraction of product for a single substrate at time t and k is the pseudo first-order cleavage rate. A relative catalytic efficiency (k_{cat}/K_{M}) of 1.0 was assigned to the internal standard peptide, APPsw. Therefore, the relative k_{cat}/K_{M} of any other substrate is determined by comparing its pseudo first-order rates of cleavage to that of the APPsw peptide. For convenience of discussion, the relative k_{cat}/K_{M} value is also referred to as “Preference Index.”

Kinetic analysis of subsite specificity using stable isotope-assisted MALDI-TOF mass spectrometry. N-acetoxy-D₀ (D₃)-succinimide was synthesized from N-hydroxysuccinimide and D₀ (D₆)-form acetic anhydride as previous described (Riggs et al., 2005). Each of the peptide mixture (P₆-1, P₇-1 and P₈-1) was equally divided. Each portion was incubated with either N-acetoxy-D₀-succinimide or N-acetoxy-D₃-succinimide in 25 mM ammonium bicarbonate, pH 7.5, for 3 hours. D₀- or D₃-aceylated modified peptide mixtures were individually diluted into 0.1 M MOPS, pH 4.0, to obtain a final concentration of 6 μM. At room temperature, an aliquot of memapsin 2 was added to the D₃-modified sample. At different incubation time points, an aliquot sample was taken out, quenched by formic acid and pooled with equal volume of the D₀-labeled sample. Combined samples were desalted with ZipTip C18 (Millipore, Billerica, Mass.). Samples of 0.5 μl were each combined with equal amount of saturated α-cyan-4 hydroxycinnamic acid matrix in 50% acetonitrile/0.1% TFA and immediately spotted in duplicate onto a MALDI sample plate and the monoisotopic mass values of the peptides were measured by Ultraflex MALDI-TOF mass spectrometer (Bruker daltonics, Bremen, Germany) operated in the reflector mode. All MALDI spectra were calibrated externally using a peptide standard. Cleavage sites were searched by calculating monoiostopic masses from prospector.ucsf.edu/prospector/mshome.htm. At each time point, relative product formation was calculated as the ratio of the reduction of substrate's signal intensity by comparing the amount of D₃ to its reference D₀. The relative product formation was plotted against time to calculate relative k_{cat}/K_{M} as described above.

Determination of k_{cat}/K_{M} of different memapsin 2's substrates. Hydrolyses of VGSC beta 2 were carried out with substrate concentration ranging from 5 μM to 150 μM in 0.1M MOPS buffer (pH 4.0), at 37° C. Initial velocity was determined by ESI LC/MS, in which the product an substrate peak areas were quantitated. The kinetic parameters, k_{cat} and K_{M} were determined from non-linear regression using Grafit software (Surrey, UK). Other memapsin 2 substrates were paired with VGSC beta 2 and subjected to competitive hydrolysis and mass spectrometry. Relative k_{cat}/K_{M} of different substrates were determined by comparing the initial hydrolysis rates to that of VGSC beta 2.

Plasmid construction and mutagenesis. APP (770 isoform) was subcloned into pSecTag vector. Different mutations flanking the β-cleavage site (P₃-P₁) APPsw, APP_{IDF} and APP_{MDL} were generated by Stratagene (La Jolla, Calif.) QuikChange Site-Directed Mutagenesis Kit and individually confirmed by DNA sequencing.

Cell culture, transfection and analysis of APP processing products. Mouse neuronal CAD cell line was cultured with DMEM/F12 media (Invitrogen, Carlsbad, Calif.) containing 10% fetal bovine serum and 2% penicillin/streptomycin. Transient trransfections were performed using Fugene HD (Roche, Sweden) according to the manufacture's instruction. 24 hours later after transfection, total cell lysate and cell media were collected and 1% of Protease inhibitor was added (Calbiochem, Gibbstown, N.J.). Aβ level in media was assayed by Aβ [1-40] Human Fluorimetric ELISA Kit (invitrogen, Carlsbad, Calif.). Conditioned media or total cell lysate from the transiently transfected cells were subjected to 10% or 10-20% tricine SDS-PAGE gels (Invitrogen, Carlsbad, Calif.), and transferred to PVDF membranes. The blots were probed with antibodies. The western blot analysis was used for determining the levels of full-length APP, APP's proteolytic products and β-actin.

### Example 2

### Results

Complete residue preference on subsites P₅-P₈. In order to assess the contribution of all subsites on memapsin 2 catalysis, one needs a complete set of subsite specificity data. Although the complete residue preferences for eight subsites, from P₄ to P_{4′} were available (Turner et al., 2001), only partial residue preferences had been determined for subsites P₅ to P₇ and there was no information on subsite P₈ since these four subsites were discovered later (Turner et al., 2004). Thus, the first task was to determine the complete residue preference in these four subsites. The strategy used for these experiments was as previously described. Briefly, the initial cleavage rates of peptide substrates in a mixture by memapsin 2 were determined using ESI-TOF mass spectrometry. The relative rates under the experimental conditions were proportional to the relative k_{cat}/K_{M} (Preference Index) values. Thus, peptide substrates differing from one another only by residues in a single subsite yielded relative preference for these residues. Peptide mixtures had the template sequence RTEEISEVNL*DAEF (* denotes the cleavage site) and contained residue variation in each of the subsites P₈, P₇, P₆, and P₅, at template residues E, E, I, and S, respectively. Each subsite position contained a mixture of all amino acids except cysteine.

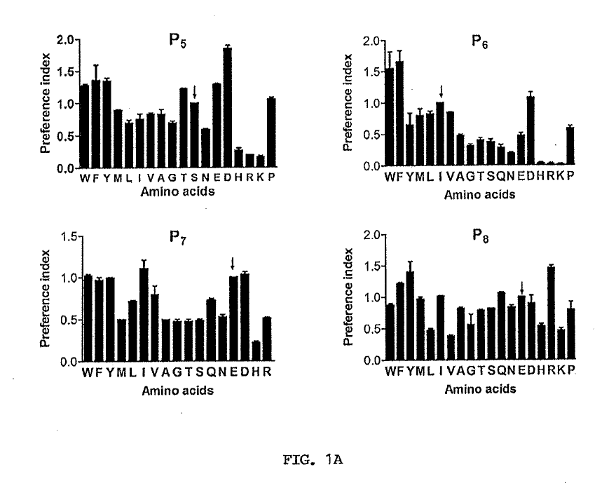

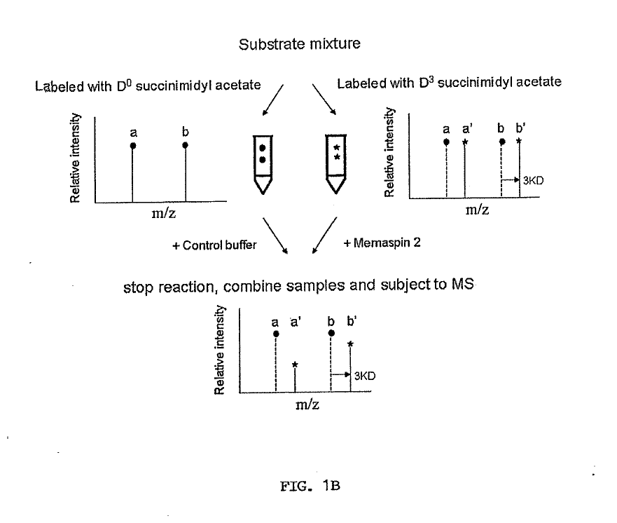

Preference Index values for residues in subsites P₈, P₇, P₆, and P₅ are shown in FIG. 1A. Among these four subsites, amino acids in P₆ have the most effect on the substrate hydrolysis and amino acid tryptophan (W) or phenylalanine (F) is most favored. In the other three subsites, the differences among the residues are less noticeable thus their plots in FIG. 1A have the appearance of a high background. In general, there is a pronounced unfavorable preference, or low Preference Index value, associated with basic amino acids, histidine (H), arginine (R), and lysine (K), in at least three subsites, P₅, P₆, and P₇. Upon comparison of the current results with the partial specificity data reported previously for subsites P₅, P₆ or P₇ (Turner et al., 2004), there is a general agreement in relative specificity. However, the positive preference of tryptophan and the negative preference of basic amino acids were much more pronounced in the first study. For this reason, the inventors used the stable-isotope-assisted MALDI-TOF mass spectrometry to confirm the current results. The experimental design (FIG. 1B) is as follows. Peptide mixtures (P₆-1, P₇-1 and P₈-1) containing selected amino acids for testing were equally divided. Each group was labeled with either N-acetoxy-D₀-succinimide or N-acetoxy-D₃-succinimide (Riggs et al., 2005). The D₃-acelyated peptides were subjected to memapsin 2 hydrolysis and mixed with equal amount of D₀-acelyated modified same peptide. The two isotopes in each sample were determined in MALDI-TOF mass spectrometer. The D₃ data represent the hydrolytic rates and the D₀ data serve as internal standard. The relative hydrolytic rates, which represent the relative k_{cat}/K_{M} values of P₆, P₇ or P₈, are shown in FIG. 1C. A comparison of data from two methods established that the relative preferences are in good agreement.

Comparison of the kinetics for peptides derived from memapsin 2 protein substrates. Amyloid precursor protein (APP) was the first identified memapsin 2 substrate. However, during the last several years, several additional substrates have been reported and some of the cleavage sites on these substrates have been identified (Table 1). Thirteen peptides of twelve-residue each were synthesized based on the sequences around these cleavage sites so that each contained subsites from P₈ to P_{4′}. This group of peptides will be referred to as the ‘substrate peptide set’. One of the peptides, VGSC-β2, was used for steady-state kinetic analysis for memapsin 2 hydrolysis resulting in k_{cat} and K_{M} values of 0.525 min⁻¹ and 36.4 μM respectively. The relative k_{cat}/K_{M} values of other twelve peptides were determined from their relative pseudo-first-order hydrolytic rates. In these experiments, peptide mixtures including the VGSC-β2 peptide were subjected to hydrolysis by memapsin 2 to determine the initial hydrolysis velocity values, under the condition [S]<<K_{M}. Since the k_{cat}/K_{M} value for VGSC-β2 peptide was known, the k_{cat}/K_{M} value of a new substrate was calculated from the ratio of its initial velocity to that of VGSC-β2 peptide (Fersht, 1985). Wild-type APP (APP_{WT}), alpha 2, 6-sialyltransferase (ST6GalI) and interleukin-1 receptor 2 (IL-1R2) are substrates with low k_{cat}/K_{M} values in the range of 1 to 5 s⁻¹M⁻¹ (Table 1). Four peptides, β1 and β3 subunit of voltage-gated sodium channel (VGSC β1, VGSC β3), P-selectin glycoprotein ligand-1 (PSGL-1) and the peptide derived from the secondary cleavage site of APP (APP_{E11}) showed even lower cleavage efficiency with k_{cat}/K_{M} values of less than 0.5 s⁻¹M⁻¹. Three peptides are significantly better substrates than APP_{WT}. The k_{cat}/K_{M} values of voltage-gated sodium channel, subunit 2 (VGSC-β2), neuregulin 1 (NRG1), and neuregulin 3 (NRG3) are between 24 and 75 s⁻¹M⁻¹. The best natural substrate is voltage-gated sodium channel, subunit 4 (VGSC-(34), with a k_{cat}/K_{M} value of almost 700 s⁻¹M⁻¹ compared to the k_{cat}/K_{M} value of the Swedish mutant of APP (APP_{sw}) value of 487 s⁻¹M⁻¹). The peptide APP_{OK1}, synthesized after choosing the most favorable amino acid from each subsite according to the subsite specificity data (Turner et al., 2001 and FIG. 1A) shows a highest k_{cat}/K_{M} among all substrates, with a value of 1761 s⁻¹M⁻¹. These results show that memapsin 2 hydrolyzes the substrate peptide set with a wide range of efficiency.

An algorithm for memapsin 2 catalytic specificity. Information on the complete subsite specificity and kinetic data from substrate peptide set permitted us to address the question of whether these data can generate a quantitative model to assess the catalytic efficiency of potential memapsin 2 cleavage sites. The inventors used the data for the substrate peptide set as a learning set to build and test an algorithm for relating the experimentally determined relative k_{cat}/K_{M} values to the calculated cleavage efficiency values. The agreement between these two set of values served to evaluate the competence of the model. In developing the algorithm, the inventors assumed that all the sidechains of the substrate are equal in accessibility by memapsin 2 and that the contribution of each sidechain in cleavage efficiency is independent of other sidechains. Also, they assumed that the contribution of each subsite to the cleavage efficiency is different from that of the other subsites, as suggested from the different stringency on residue specificity in different subsites. These assumptions led us to a equation similar to a weighted geometric mean of the various specificities since the inventors expected the effects of the individual subsites to be multiplicative. The resulting equation is:

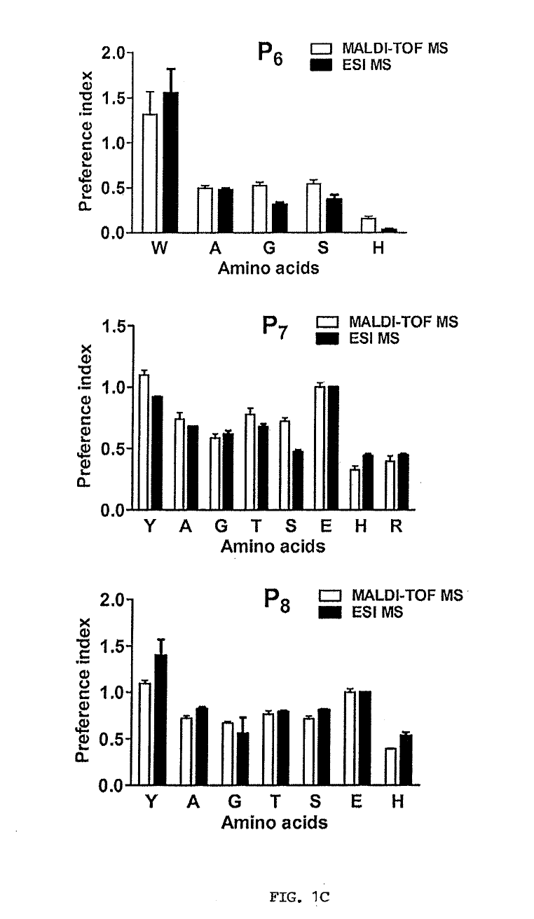

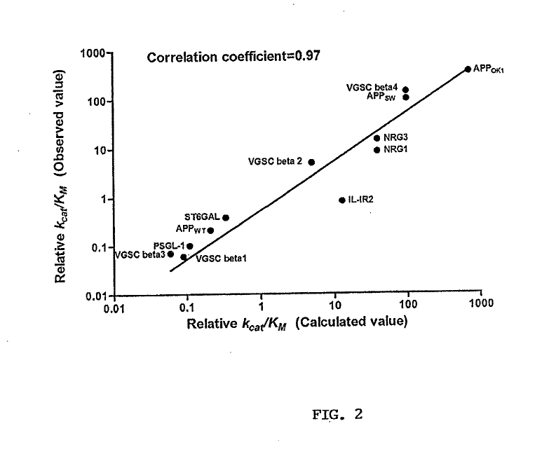

Q=Exp(Σwᵢ ln aᵢ)

where Q is the arbitrary value for memapsin 2 cleavage efficiency, aᵢ is the experimentally determined relative k_{cat}/K_{M} value (Table 5) of the amino acid at Pᵢ subsite position and wᵢ is the weighting factor of that particular subsite. The wᵢ values were determined by non-linear regression to achieve a maximal correlation coefficient value between the Q values and the actual kinetic data of the substrate peptide set. The optimized wᵢ values are shown in Table 2 and corresponding Q values for the substrate peptide set are shown in Table 1. During the optimization process, the inventors found that only six subsites, P₄ to P_{2′}, significantly influenced the calculated Q values; thus, the outside subsites were dropped from the further calculations. A plot of the Q values and the relative k_{cat}/K_{M} data showed a linear correlation (FIG. 2) with a correlation coefficient of 0.97.

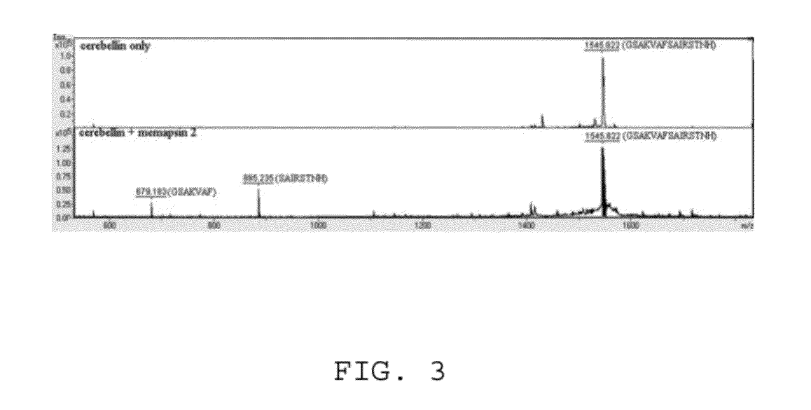

To test this algorithm, the inventors selected a 15-residue peptide cerebellin (GSAKVAFSAIRSTNH), which is not a natural substrate of memapsin 2. This peptide was chosen because it is small enough to be unbiased in specific tertiary structures yet contains enough residues to be recognized by multiple subsites of memapsin 2. The application of the algorithm predicted a distinct cleavage site at the Phe-Ser bond (Table 3) with a k_{cat}/K_{M} value of 0.24 Analysis of hydrolytic products of cerebellin by memapsin 2 in MALD-TOF mass spectrometry showed essentially two products with mass of 679.18 Da and 885.24 Da (FIG. 3) which are assigned to the fragment GSAKVAF and SAIRSTNH, the N-terminal and C-terminal products generated from the predicted cleavage site respectively. The k_{cat}/K_{M} value for the cleavage of this site determined was 0.14 s⁻¹M⁻¹. This value is slightly lower than the prediction which may be due to the presence of low level of conformational strain in a larger peptide substrate. Overall, these results confirmed the predicted cleavage site using the algorithm.

Design an APP mutant for maximal production of amyloid-beta. The results above show that the mutations of P₂ Lys and P₁ Met in APP_{WT} to P₂ Asn and P₁ Leu respectively (APP_{SW}) increased the k_{cat}/K_{M} value by 477-fold (Table 1). Since the P′ residues are not changed, both APP_{WT} and APP_{SW} produce the same amyloid-β (Aβ) peptides and this greatly enhanced production is attributed as the cause of an early onset of Alzheimer's Disease in APP_{SW} mutation. With the availability of the algorithm described above, it was of interest to design a highly efficient memapsin 2 cleaving APP mutant with new residue mutations only on the P subsites, thus it would still produce the native Aβ. The algorithm predicted that the mutation of residues in APP_{WT} from P₃ Val, P₂ Lys, and P₁ Met to P₃ Ile, P₂ Asp, and P₁ Phe, respectively, (APP_{IDF}) would increase the k_{cat}/K_{M} value by about 849-fold, about 1.7-times higher than that for APP_{SW}. To investigate the ability of APP_{IDF} to generate Aβ in the cells, the inventors mutated these three residues in APP_{WT}, transfected the expression vector of APP_{IDF} into mouse neuronal CAD cells, and determined degradation products of APP in cells and culture medium. Another APP variant with P₃ Met, P₂ Asp, and P₁ Phe, APP_{MDF}, was also included in this study. APP_{MDF} has a predicted k_{cat}/K_{M} value about 18-times that for APP_{WT}, so it may serve as comparison for the Aβ response in the cells.

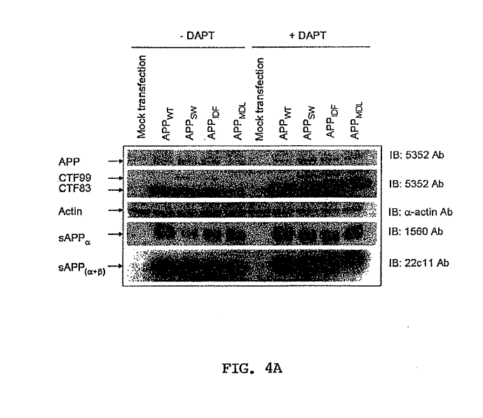

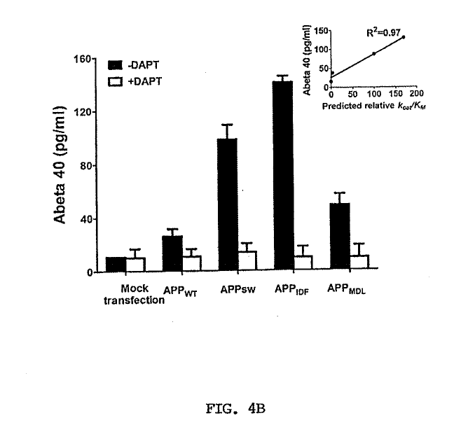

The Western blot for APP indicated that the expression levels of APP_{WT}, APP_{SW}, APP_{IDF} and APP_{MDF} were about the same (FIG. 4A, top panel). ELISA determination of Aβ in the culture media indicated that APP_{SW} cells produced about four-fold of Aβ than did APP_{WT} cells and the cells expressing APP_{IDF} produced about 40% more Aβ than APP_{SW} cells (FIG. 4B). As expected, Aβ produced by APP_{MDF} was between APP_{WT} and APP_{SW}. A plot of AO and predicted relative k_{cat}/K_{M} values of four APP clones showed a good linear correlation (FIG. 4B, inset). APP_{IDF} and APP_{SW} cells were also similar in cellular processing characteristics. Both revealed a detectable accumulation of APP C-terminal fragment of 99 residues (CTF99), the direct product of memapsin 2 cleavage, which is not visible in APP_{WT} cells (FIG. 4A, second panel, left lanes). CTF99 is increased in both APP_{IDF} and APP_{SW} cells when γ-secretase inhibitor DAPT slowed its degradation (FIG. 4A, second line, right lanes). As expected, APP ectodomain fragment from α-secretase cleavage, sAPPα, decreased in both APP_{IDF} and APP_{SW} cells as compared to that in APP_{WT} cells (FIG. 4A, fourth panel). Taken together, the above results indicate that APP_{IDF} produces a higher level of Aβ than that for APPsw in a neuronal cell line and its degradation pathways are mediated through three secretases.

### Example 3

### Discussion

The determination of subsite specificity of aspartic proteases usually requires many kinetic analyses. Since most of these proteases have eight or more subsites and have non-stringent specificity, very few subsite specificity of these enzymes have been completely determined. For memapsin 2, the residue preference, expressed as relative k_{cat}/K_{M} values, is now known for twelve subsites, from P₈ to P₄′. Therefore, these data on memapsin 2 represent the first complete kinetic assessment of the subsite preference of an aspartic protease and offers a new opportunity to dissect the influence of subsite residues on its hydrolytic activity. The test of the general applicability of the subsite specificity information was done in two parts. The relative k_{cat}/K_{M} values of thirteen peptides of memapsin 2 substrates, or study set, were first determined. Using these data as guide, an algorithm was developed to quantitatively assess any new site for memapsin 2 cleavage activity. The predicted memapsin 2 activity on the study set peptides and the experimental data produced a correlation coefficient of about 0.97, confirming the predictive potential of the algorithm. In addition, test peptides not naturally hydrolyzed by memapsin 2 substantiated the prediction calculations of the algorithm model.

The inventors have used the algorithm to predict memapsin 2 cleavage activity of several proteins of interest. Firstly, four peptides containing sequences from the reported memapsin 2 substrates (Table 1, numbers 3, 5, 11 and 12) were very poor substrates and the kinetic data were obtained for them using high protease concentration and prolonged incubation. Considering that the relative k_{cat}/K_{M} value of APPwt is only a few-fold of the values for these four peptides, it seems to suggest that the functions of memapsin 2 do not require substrates with highly favorable bonds. Part of the reasons of this may be due to that both enzyme and substrates are membrane anchored so the substrate is well positioned to be cleaved. This line of argument is supported by the observation that the memapsin 2 cleaved bonds in these protein substrates are located in a region from 11 to 29 amino acid residues from the membrane (Table 1). These data also clearly show that subsite specificity is the main factor for cleavage efficiency, once the substrate is in the protease's effective range. For example, the cleavage position of APPwt and APPsw are both 29 residues from the membrane yet differ in hydrolytic efficiency by near 500-times. Another interesting point related to the above discussion is that memapsin 2 cleavage sites in APPwt (Table 1, number 1) and APPE11 (Table 1, number 13) are present on the same APP molecule, thus should be competing cleavage sites under physiological conditions. The cleavage of APPE 11 precludes the formation of Aβ, instead, it produces a shorter peptide, Glu11-Aβ, after the γ-secretase cleavage (Liu et al., 2002). Based on the relative k_{cat}/K_{M} of these two sites, the ratio of Aβ to Glu11-Aβ would be about 50 to 1 in cells producing APPwt. However, in cells producing APPsw, this ratio would be about 25,000 to 1, greatly diminished the production of Glu11-Aβ and its possible physiological roles.

Secondly, two APP homologues, APLP1 and APLP2 have been shown by several laboratories to be cleaved by memapsin 2 (Li and Sudhof, 2004; Pastorino et al., 2004; Walsh et al., 2007). However, the actual cleavage sites have not been determined. The inventors have used the algorithm to predict the potential memapsin 2 cleavage sites on these proteins. To narrow down the region for calculations, an assumption was made that the cleavage site on these proteins will be within or near the range of distance from membrane, from 11 to 31 amino acid residues, as in other memapsin 2 protein substrates (Table 1). Since memapsin 2 is also membrane anchored, it is reasonable to assume that it has an effective radius for its activity on membrane anchored protein substrates. The inventors applied the algorithm calculation to both proteins for the region of 55 residues adjacent to the membrane in the ectodomains. For APLP2, two potential cleavage sites (sites 1 and 2 in Table 4) were predicted at 40 and 34 residues from the membrane. The estimated memapsin 2 cleavage efficiency are about the same (site 1 and 2) as that of the β-site of APP_{WT} (Table 1). Since APP_{WT} is an established substrate, these two sites are the primary possibilities in APLP2 cleavage by memapsin 2. Sites 3 and 4 (Table 4) have the next highest predicted kinetic values, which are, however, about 50- to 100-times lower than the values for sites 1 and 2. Thus, memapsin 2 cleavage of sites 3 and 4 seems less probably even though these sites are located in the effective cleavage range. For APLP1, the algorithm predicted no efficient cleavage site. The site with the highest kinetic value is only 1/4,000 in cleavage efficiency as compared to the β-site of APP_{WT} (Table 4). These results suggest that APLP1 is not an effective substrate of memapsin 2.

Thirdly, prostaglandin E2 synthetase 2 (PGES2), a membrane protein, has been shown recently to be cleaved by memapsin 2 (Kihara et al., 2010). The proposed cleavage site, however, is extremely unfavorable (site 14, Table 4) and is unlikely to be cleaved by memapsin 2. Two nearby sites (sites 15 and 16, Table 4) have much better values for cleavage preference, especially the second one. They are more likely to be the probable sites for memapsin 2 processing. The usefulness of the current algorithm prediction is also illustrated in the case of memapsin 2 cleavage site in α2, 6-sialotransferase. The cleavage site initially reported (site 18, Table 4) (Kitazume et al., 2001) was three residues away from the actual cleavage site later determined (site 17, Table 4) Kitazume et al., 2005). The predicted kinetic values (Table 4) show that site 17 is favorable and site 18 is extremely unfavorable for memapsin 2 cleavage.

The algorithm described here was developed based on the assumption that the recognition of each sidechain by a protease subsite is independent and the peptide substrates have random conformation in solution. The very high correlation between the predicted Preference Constants (relative k_{cat}/K_{M}) and the actual data derived from in vitro experiments appears to support the assumption. In vivo substrates of memapsin 2 are proteins which conceivably may retain some conformation in the peptide strands near the cleavage sites and may differ from the in vitro rates. However, the fact that substrate analogues bind to the memapsin 2 active site in extended conformation argues for an extended, denatured state of the peptide strands at least locally near the cleavage sites. Such a ‘local denaturation’ of the cleavage sites could be facilitated by the acidic environment inside of the endosomal vesicles where the majority of memapsin 2 activity is manifested (Koo and Squazzo, 1994; Hartmann et al., 1997).

The kinetic data on natural substrates of memapsin 2 offers an interesting range of hydrolytic efficiency of about 35,000-fold variation (Table 1). The wild-type APP_{WT}, which is the best established physiological substrate of memapsin 2, is in fact among the substrates with relatively low hydrolytic efficiency by memapsin 2. APP with Swedish mutations, APP_{SW}, which replace P₂ Lys and P₁ Met of APP_{WT} with Asn and Leu respectively and manifesting an early onset form of Alzheimer's disease, increased the k_{cat}/K_{M} value by 479-times. These comparisons argue for the hypothesis that the structural mutations to attain the highest cleavage efficiency of APP as a memapsin 2 substrate have not been subjected to survival selection in evolution. This may be because other criteria, such as regulation of Aβ production, are more important criteria for evolutionary selection. The best hydrolyzing natural substrate studied is voltage gated sodium channel, subunit 4, which has a k_{cat}/K_{M} value 1.43-times higher than that of APP_{SW}. Peptides from four of the reported substrates were extremely poor substrate (Table 1) of memapsin 2 and extensive incubation with the protease produced negligible amounts of hydrolysis.

The design of APP_{IDF} further demonstrated the potential application of the algorithm model. The predicted hydrolytic efficiency of APP_{IDF} by memapsin 2 is 1.7-fold of that for APP_{SW}. In cellular experiments, the inventors observed that the production of Aβ from APP_{IDF} up to 1.5-fold that from APP_{SW}. Up to now, APP_{SW} has been the APP mutant that produces the highest amount of AP and its sequence has been used in peptide substrates for memapsin 2 assays. APP_{SW} has also been used to produce a number of transgenic mouse strains (Hsiao et al., 1996; Jankowsky et al., 2001) that manifest both brain amyloid plaques and loss of cognitive functions upon aging. These mouse strains are widely used as experimental models for Alzheimer's disease in human. The current results indicate that peptides containing APP_{IDF} would be more efficient substrates for memapsin 2 than those containing APP_{SW} sequences. These results also suggest that it would be of interest to study transgenic mouse strains with the APP_{IDF} mutations as animal models of AD. The probability of a clinical observation of an early onset of Alzheimer's disease with APP_{IDF} mutations is probably very small since five mutations need to occur for the conversion of APP_{WT} to APP_{IDF}, as compared with the formation of APPsw would need only two mutation steps.

### Example 4

### Computer System

The methods described herein may be implemented in computer systems configured to perform the steps recited above. For example, the computer sytem may have computer memory for holding information relating to a site comprising an amino acid sequence of at least five residues in length, wherein consecutive residues of said sequence are assigned as subsites P₃, P₂, P₁, P₁′, and P₂′ in an N- to C-terminal order, wherein cleavage occurs between P₁ and P₁′. The computer memory may be volatile or non-volatile memory and may be located remotely. For example, a server may hold information relating to the sites to be evaluated and the server may be accessible over the internet by a PC that predicts a relative memapsin 2 cleavage efficiency for the site based on that information.

The systems may also include a computer processor configured to read the information relating to the site from the computer memory and to obtain a cleavage preference value for each of subsites P₃, P₂, P₁, P₁′, and P₂′ based on the formula Q=Exp(Σwᵢ ln aᵢ) as described above. The computer processor may be a general purpose processor such as those found in desktop and laptop PCs or may be a dedicated processor, such as a Digital Signal Processor (DSP), for performing the methods described herein.

### Example 5

### Computer Program Products

Also disclosed are computer program products that include a computer readable medium having computer usable program code. The code can be executed by a processor, such as a processor described in connection with Example 4, for performing operations for processing data. For example, the code can be executed for predicting a relative memapsin 2 cleavage efficiency for a site in a peptide or polypeptide sequence as described herein. The computer program product may be a hard drive, a Compact Disk (CD), a DVD, a floppy disk drive, a tape drive, a flash drive, or similar medium for storing computer code. The computer usable program code, when executed, may carry out the methods described herein.

TABLE 1
Comparison of sequence and kinetic properties of different memapsin 2 substrates

| | | | | | | | | | | | | | | | | Relative k_{cat}/K_{M}^{d} | |
|---|---|---|---|---|---|---|---|---|---|---|---|---|---|---|---|---|---|
| | Sequence^{b} | | | | | | | | | | | | | Cleavage site from | k_{cat}/K_{M} | Observed | Calculated |
| Substrate^{a} | P₈ | P₇ | P₆ | P₅ | P₄ | P₃ | P₂ | P₁ | *^{c} | P₁′ | P₂′ | P₃′ | P₄′ | membrane (a.a.) | (s⁻¹M⁻¹) | value | value |
| (1) APP_{WT} | E | E | I | S | E | V | K | M | | D | A | E | F | 29 | 1.02 ± 0.05 | 0.21 | 0.21 |
| (2) APP_{SW} | E | E | I | S | E | V | N | L | | D | A | E | F | 29 | 486.55 ± 82.2 | 100 | 100 |
| (3) VGSC-β1 | S | V | V | K | K | I | H | L | | E | V | V | D | 16 | 0.30 ± 0.02 | 0.06 | 0.09 |
| (4) VGSC-β2 | R | G | H | G | K | I | Y | L | | Q | V | L | L | 13 | 24.30 ± 2.38 | 4.99 | 5.04 |
| (5) VGSC-β3 | N | V | S | R | E | F | E | F | | E | A | H | R | 31 | 0.33 ± 0.13 | 0.07 | 0.06 |
| (6) VGSC-β4 | N | N | S | A | T | I | F | L | | Q | V | V | D | 12 | 695.88 ± 97.93 | 143.02 | 99.42 |
| (7) ST6GalI | S | D | Y | E | A | L | T | L | | Q | A | K | E | 11 | 1.85 ± 0.37 | 0.38 | 0.34 |
| (8) IL-IR2 | V | V | H | N | T | L | S | F | | Q | T | L | R | 15 | 4.00 ± 0.14 | 0.82 | 12.99 |
| (9) NRG1 | Y | K | H | L | G | I | E | F | | M | E | A | E | 11 | 41.39 ± 7.74 | 8.51 | 39.79 |
| (10) NRG3 | T | D | H | L | G | I | E | F | | M | E | S | E | 11 | 72.07 ± 9.87 | 14.81 | 39.79 |
| (11) PSGL-1 | I | P | M | A | A | S | N | L | | S | V | N | Y | 17 | 0.48 ± 0.02 | 0.10 | 0.11 |
| (12) APP_{EI1} | E | F | R | H | D | S | G | Y | | E | V | H | H | 19 | 0.02 ± 0.01 | 0.004 | 3.9 × 10⁻⁶ |
| (13) APP_{OK1} | Y | I | W | D | E | I | D | L | | M | V | L | D | 29 | 1760.59 ± 124.52 | 361.85 | 722.24 |

^{a}(1) App_{WT} represents wild type APP. (2) APP_{SW} represents Swedish APP. (3)-(6) VGSC-β1, β2, β3 and β4 represent β1 to β4 subunits of voltage-gated sodium channels (7) ST6GalI represents α-2,6-sialyltransferase. (8) IL-IR2 represents interleukin-1 receptor 2. (9) NRG1 represents neuregulin 1. (10) NRG3 represents neuregulin 3. (11) PSGL-1 represents P-selectin glycoprotein ligand-1. (12) APP_{EI1} represents memapsin 2 alternative cleavage site on APP. (13) APP_{OK1} is not an natural substrate and synthesized by choosing the most favorable amino acid from each subsite according to the subsite specificity data (ref. (27) and FIG. 1a)
^{b}Amino acid residues are shown in one-letter code.
^{c}*denotes the cleavage site.
^{d}Relative k_{cat}/K_{M} of APP_{SW} is arbitrarily assigned as 100, the relative k_{cat}/K_{M} values of other substrates are normalized to APP_{SW}.

TABLE 2
Weighting factor for P₄ to P₂′ subsite

| W₄ | W₃ | W₂ | W₁ | W₁′ | W₂′ |
|---|---|---|---|---|---|
| 0.89 | 3.50 | 1.02 | 6.26 | 0.38 | 1.09 |

TABLE 3
Comparison of possible cleavage sites in cerebellin

| Possible cleavage sites^{a} | Possible Peptides | mass after cleavage | Predicted relative k_{cat}/K_{M}^{b} |
|---|---|---|---|
| GSAKVAFSAIR * STNH | 1106.63 | 458.20 | 1.34 × 10⁻¹⁵ |
| GSAKVAFSAI * RSTNH | 950.53 | 614.30 | 1.80 × 10⁻¹⁵ |
| GSAKVAFSA * IRSTNH | 837.45 | 727.38 | 2.75 × 10⁻¹⁶ |
| GSAKVAFS * AIRSTNH | 766.41 | 798.42 | 3.22 × 10⁻⁸ |
| GSAKVAF * SAIRSTNH | 679.38 | 885.45 | 0.24 |
| GSAKVA * FSAIRSTNH | 532.31 | 1032.52 | 2.34 × 10⁻¹⁵ |
| GSAKV * AFSAIRSTNH | 461.27 | 1103.56 | 1.74 × 10⁻¹⁴ |
| GSAK * VAFSAIR STNH | 362.20 | 1202.63 | 1.30 × 10⁻¹⁴ |

^{a}Amino acid residues are shown in one-letter code; *represent the possible cleavage site.
^{b}Relative k_{cat}/K_{M} APPsw is arbitrarily assigned as 100, the predicted relative k_{cat}/K_{M} values of different possible cleavage site are normalized to APPsw.

TABLE 4
Comparison of possible cleavage sites in APLP1, APLP2, PGES-2 and
ST6GalI by memapsin 2

| | | Predicted | Distance |
|---|---|---|---|
| | | relative | from |
| Protein^{a} | Sequence and possible cleavage site^{b} | k_{cat}/K_{M}^{c} | membrane |
| | 1 2 3 4 5 6 | | |
| | ▾ ▾ ▾ ▾ ▾ ▾ | | |
| APLP2 | KVDENM VIDETL DVKEM IF NAERVGGL EEERESVGPL REDFSLSSS | 1. 0.23 | 40 |
| | | 2. 0.18 | 34 |
| | | 3. 1 × 10⁻³ | 29 |
| | | 4. 4 × 10⁻³ | 27 |
| | | 5. 7 × 10⁻⁷ | 19 |
| | | 6. 1 × 10⁻⁷ | 9 |
| | 7 8 9 10 11 12 | | |
| | ▾ ▾ ▾ ▾ ▾ ▾ | | |
| APLP1 | PEKEKM NPL EQY ERKVNASVPRGF PF HSSEIQRDEL APAGTGVSRE | 7. 1 × 10⁻⁶ | 40 |
| | | 8. 6 × 10⁻⁹ | 37 |
| | | 9. 3 × 10⁻⁶ | 34 |
| | | 10. 3 × 10⁻⁹ | 22 |
| | | 11. 3 × 10⁻⁵ | 20 |
| | | 12. 5 × 10⁻⁵ | 10 |
| | 13 14 15 16 | | |
| | ▾ ▾ ▾ ▾ | | |
| mPGES-2 | HLRAQDL HA ERSAAQL SL SS | 13. 1 × 10⁻⁴ | ^{d} |
| | | 14. 6 × 10⁻¹⁴ | ^{d} |
| | | 15. 0.08 | ^{d} |
| | | 16. 36 | ^{d} |
| | 17 18 | | |
| | ▾ ▾ | | |
| ST6GalI | SDYEALTL QAK EFQ | 17. 0.3 | 11 |
| | | 18. 1 × 10⁻¹⁶ | 14 |

^{a}APLP1 and APLP2 represent Amyloid beta (A4) precursor-like protein 1 and 2; mPGES-2 represents Membrane-associated prostaglandin E2 synthase-2; ST6GAL represents α-2,6-sialyltransferase.
^{b}Amino acid residues are shown in one-letter code; represent the possible cleavage site.
^{c}Relative k_{cat}/K_{M} of APPsw is arbitrarily assigned as 100, the predicted relative k_{cat}/K_{M} values of different possible cleavage site are normalized to APPsw.
^{d}mPGES-2 is a membrane-associated protein instead of transmembrane protein like the other proteins in this table.

TABLE 5
Catalytic Efficiency Residue Preference: Memapsin 2

| | Upstream subsites | | | | Downstream subsites | | | |
|---|---|---|---|---|---|---|---|---|
| | P₄ | P₃ | P₂ | P₁ | P_{1′} | P_{2′} | P_{3′} | P_{4′} |
| W | 0.19 | 0.01* | 0.01 | 0.01* | 0.03 | 0.02 | 1.85 | 1.22 |
| F | 0.17 | 0.17 | 0.69 | 0.88 | 0.14 | 0.92 | 0.95 | 1.00 |
| Y | 0.05 | 0.02 | 0.58 | 0.29 | 0.37 | 0.61 | 0.86 | 1.02 |
| M | 0.45 | 0.36 | 0.97 | 0.54 | 1.47 | 0.73 | 0.82 | 1.00 |
| L | 0.25 | 1.23 | 0.59 | 1.00 | 0.30 | 0.94 | 1.24 | 0.81 |
| I | 0.11 | 1.37 | 0.01* | 0.01* | 0.13 | 1.38 | 1.24 | 0.78 |
| V | 0.17 | 1.00 | 0.01* | 0.01* | 0.20 | 1.41 | 1.79 | 0.85 |
| A | 0.12 | 0.39 | 0.34 | 0.02 | 1.00 | 1.00 | 0.78 | 0.73 |
| G | 0.39 | 0.02 | 0.02 | 0.04 | 0.04 | 0.16 | 0.69 | 0.68 |
| T | 0.24 | 0.38 | 0.01* | 0.16 | 0.24 | 0.87 | 1.15 | 0.81 |
| S | 0.14 | 0.22 | 0.50 | 0.07 | 0.67 | 0.48 | 0.66 | 0.69 |
| Q | 0.85 | 0.05 | 0.17 | 0.01* | 1.09 | 0.13 | 0.63 | 0.74 |
| N | 0.43 | 0.01* | 1.00 | 0.02 | 0.04 | 0.03 | 0.47 | 0.04 |
| E | 1.00 | 0.63 | 0.53 | 0.01* | 1.32 | 0.96 | 1.00 | 1.29 |
| D | 0.64 | 0.11 | 1.22 | 0.06 | 0.82 | 0.02 | 1.05 | 1.34 |
| H | 0.29 | 0.53 | 0.01* | 0.02 | 0.01* | 0.01* | 0.17 | 0.21 |
| R | 0.24 | 0.01* | 0.01* | 0.01* | 0.06 | 0.01* | 0.76 | 0.24 |
| K | 0.01* | 0.29 | 0.10 | 0.01* | 0.06 | 0.02 | 0.78 | 0.10 |
| P | 0.25 | 0.37 | 0.01* | 0.01* | 0.01* | 0.01* | 0.02 | 0.01* |

*The relative Kcat/Km of that peptide in the mixture is too low to be determined. We arbitrarily assume the relative preference as 0.01.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### IX. REFERENCES

The following references, to the extent that they provide exemplary procedural or other details supplementary to those set forth herein, are specifically incorporated herein by reference.
Ermolieff et al., Biochemistry, 39:16263, 2000.
Fersht, In: Enzyme structure and mechanism, 2^{nd} Ed., W. H. Freeman, NY, 1985.
Ghosh et al., Neurotherapeutics, 5:399-408, 2008.
Hartmann et al., Nat. Med., 3:1016-20, 1997.
Hong et al., Biochemistry, 41:10963-7, 2002.
Hong et al., Science, 290:150-3, 2000.
Hsiao et al., Science, 274:99-102, 1996.
Hu et al., Nat. Neurosci., 9:1520-5, 2006.
Jankowsky et al., Biomol. Eng., 17:157-65, 2001.
Kamenetz et al., Neuron., 37:925-37, 2003.
Kang et al., Nature, 325:733-736, 1987.
Kihara et al., Biochem. Biophys. Res. Commun., 393:728-733, 2010.
Kim et al., Nat. Cell Biol., 9:755-64, 2007.
Kitazume et al., J. Biol. Chem., 280:8589-95, 2005.
Kitazume et al., Proc. Natl. Acad. Sci. USA, 98:13554-9, 2001.
Koo and Squazzo, J. Biol. Chem., 269:17386-9, 1994.
Kuhn et al., J. Biol. Chem., 282:11982-95, 2007.
Lauren et al., Nature, 457:1128-32, 2009.
Li and Sudhof, J. Biol. Chem., 279:10542-50, 2004.
Lichtenthaler et al., J. Biol. Chem., 278:48713-9, 2003.
Lin et al., Proc. Natl. Acad. Sci. USA, 97:1456-60, 2000.
Liu et al., Biochemistry, 41:3128-36, 2002.
Miyazaki et al., Biochem. Biophys. Res. Commun., 361:43-8, 2007.
Nikolaev et al., Nature, 457:981-9, 2009.
Pastorino et al., Mol. Cell Neurosci., 25:642-9, 2004.
Riggs et al., J. Chromatogr B Analyt. Technol. Biomed. Life Sci., 817:89-96, 2005.
Selkoe, Nature, 399:A23-31, 1999.
Selkoe, Physiol. Rev., 81:741-766, 2001.
Turner et al., Biochemistry, 40:10001-6, 2001.
Turner et al., Biochemistry, 43, 2004.
von Arnim et al., J. Biol. Chem., 280:17777-85, 2005.
Walsh et al., Biochem. Soc. Trans., 35:416-20, 2007.
Willem et al., Science, 314:664-6, 2005.
Wong et al., J. Biol. Chem., 280:23009-17, 2005.

What is claimed is:

1. A method of predicting a relative memapsin 2 cleavage efficiency for a site in a peptide or polypeptide sequence comprising:
(a) providing a site comprising an amino acid sequence of at least five residues in length, wherein consecutive residues of said sequence are assigned as subsites P₃, P₂, P₁, P₁′, and P₂′ in an N- to C-terminal order, wherein cleavage occurs between P₁ and P₁′;
(b) obtaining a cleavage preference value for each of subsites P₃, P₂, P₁, P₁′, and P₂′ based on the following formula:
Q=Exp(Σwᵢ ln aᵢ)
wherein Q is value for memapsin 2 cleavage efficiency, aᵢ is the relative k_{cat}/K_{M} value for Pᵢ from the following chart:

| | |
|---|---|
| Upstream | Downstream |
| | P₄ | P₃ | P₂ | P₁ | P_{1′} | P_{2′} |
|---|---|---|---|---|---|---|
| W | 0.19 | 0.01* | 0.01 | 0.01* | 0.03 | 0.02 |
| F | 0.17 | 0.17 | 0.69 | 0.88 | 0.14 | 0.92 |
| Y | 0.05 | 0.02 | 0.58 | 0.29 | 0.37 | 0.61 |
| M | 0.45 | 0.36 | 0.97 | 0.54 | 1.47 | 0.73 |
| L | 0.25 | 1.23 | 0.59 | 1.00 | 0.30 | 0.94 |
| I | 0.11 | 1.37 | 0.01* | 0.01* | 0.13 | 1.38 |
| V | 0.17 | 1.00 | 0.01* | 0.01* | 0.20 | 1.41 |
| A | 0.12 | 0.39 | 0.34 | 0.02 | 1.00 | 1.00 |
| G | 0.39 | 0.02 | 0.02 | 0.04 | 0.04 | 0.16 |
| T | 0.24 | 0.38 | 0.01* | 0.16 | 0.24 | 0.87 |
| S | 0.14 | 0.22 | 0.50 | 0.07 | 0.67 | 0.48 |
| Q | 0.85 | 0.05 | 0.17 | 0.01* | 1.09 | 0.13 |
| N | 0.43 | 0.01* | 1.00 | 0.02 | 0.04 | 0.03 |
| E | 1.00 | 0.63 | 0.53 | 0.01* | 1.32 | 0.96 |
| D | 0.64 | 0.11 | 1.22 | 0.06 | 0.82 | 0.02 |
| H | 0.29 | 0.53 | 0.01* | 0.02 | 0.01* | 0.01* |
| R | 0.24 | 0.01* | 0.01* | 0.01* | 0.06 | 0.01* |
| K | 0.01* | 0.29 | 0.10 | 0.01* | 0.06 | 0.02 |
| P | 0.25 | 0.37 | 0.01* | 0.01* | 0.01* | 0.01* |

and wᵢ is the weighing factor of each Pᵢ as shown below:

| W4 | W3 | W2 | W1 | W1′ | W2′ |
|---|---|---|---|---|---|
| 0.89 | 3.50 | 1.02 | 6.26 | 0.38 | 1.09 |

2. The method of claim 1, further comprising assessing a cleavage preference for subsite P₄ based on the preference chart, wherein subsite P₄ is N-terminal to subsite P₃, and creating a predicted k_{cat}/K_{M} for said site based on values from step (b).

3. The method of claim 1, wherein said peptide or polypeptide is a known substrate for memapsin 2.

4. The method of claim 1, wherein said peptide or polypeptide is not a known substrate for memapsin 2.

5. The method of claim 1, wherein said peptide or polypeptide is a disease polypeptide.

6. The method of claim 1, wherein said site is located in a peptide.

7. The method of claim 1, wherein said site is located in a polypeptide.

8. The method of claim 1, further comprising subjecting said peptide or polypeptide comprising said site to cleavage by memapsin 2.

9. The method of claim 1, further comprising modifying at least one residue in said site.

10. The method of claim 9, further comprising performing steps (a) and (b) of claim 1 on the modified site.

11. The method of claim 1, further comprising providing a site that is modified in at least one residue as compared to the site provided in step (a), and performing step (b) on the modified site.

12. The method of claim 11, further comprising preparing a peptide or polypeptide comprising the modified site.

13. The method of claim 12, further comprising subjecting the modified peptide or polypeptide comprising said site to cleavage by memapsin 2.

14. The method of claim 1, further comprising providing said peptide or polypeptide to a subject.

15. The method of claim 8, further comprising determining an actual k_{cat}/K_{M} for the site.

16. The method of claim 12, further comprising providing the modified peptide or polypeptide to a subject.

17. The method of claim 13, further comprising determining an actual k_{cat}/K_{M} for the modified site.

18. The method of claim 1, wherein step (b) employs a computer to generate said cleavage preference value.

19. A system for predicting a relative memapsin 2 cleavage efficiency for a site in a peptide or polypeptide sequence the system comprising:
(a) computer memory configured to hold information relating to a site comprising an amino acid sequence of at least five residues in length, wherein consecutive residues of said sequence are assigned as subsites P₃, P₂, P₁, P₁′, and P₂′ in an N- to C-terminal order, wherein cleavage occurs between P₁ and P₁′; and
(b) a computer processor configured to read the information relating to the site from the computer memory and to obtain a cleavage preference value for each of subsites P₃, P₂, P₁, P₁′, and P₂′ based on the following formula:
Q=Exp(Σwᵢ ln aᵢ)
wherein Q is value for memapsin 2 cleavage efficiency, aᵢ is the relative k_{cat}/K_{M} value for Pᵢ from the following chart:

| | |
|---|---|
| Upstream | Downstream |
| | P₄ | P₃ | P₂ | P₁ | P_{1′} | P_{2′} |
|---|---|---|---|---|---|---|
| W | 0.19 | 0.01* | 0.01 | 0.01* | 0.03 | 0.02 |
| F | 0.17 | 0.17 | 0.69 | 0.88 | 0.14 | 0.92 |
| Y | 0.05 | 0.02 | 0.58 | 0.29 | 0.37 | 0.61 |
| M | 0.45 | 0.36 | 0.97 | 0.54 | 1.47 | 0.73 |
| L | 0.25 | 1.23 | 0.59 | 1.00 | 0.30 | 0.94 |
| I | 0.11 | 1.37 | 0.01* | 0.01* | 0.13 | 1.38 |
| V | 0.17 | 1.00 | 0.01* | 0.01* | 0.20 | 1.41 |
| A | 0.12 | 0.39 | 0.34 | 0.02 | 1.00 | 1.00 |
| G | 0.39 | 0.02 | 0.02 | 0.04 | 0.04 | 0.16 |
| T | 0.24 | 0.38 | 0.01* | 0.16 | 0.24 | 0.87 |
| S | 0.14 | 0.22 | 0.50 | 0.07 | 0.67 | 0.48 |
| Q | 0.85 | 0.05 | 0.17 | 0.01* | 1.09 | 0.13 |
| N | 0.43 | 0.01* | 1.00 | 0.02 | 0.04 | 0.03 |
| E | 1.00 | 0.63 | 0.53 | 0.01* | 1.32 | 0.96 |
| D | 0.64 | 0.11 | 1.22 | 0.06 | 0.82 | 0.02 |
| H | 0.29 | 0.53 | 0.01* | 0.02 | 0.01* | 0.01* |
| R | 0.24 | 0.01* | 0.01* | 0.01* | 0.06 | 0.01* |
| K | 0.01* | 0.29 | 0.10 | 0.01* | 0.06 | 0.02 |
| P | 0.25 | 0.37 | 0.01* | 0.01* | 0.01* | 0.01* |

and wᵢ is the weighing factor of each P as shown below:

| W4 | W3 | W2 | W1 | W1′ | W2′ |
|---|---|---|---|---|---|
| 0.89 | 3.50 | 1.02 | 6.26 | 0.38 | 1.09 |

20. A computer program product comprising a computer readable medium having computer usable program code executable to perform operations for processing data, the operations of the computer program product comprising the steps of claim 1.


SEQUENCE LISTING (18 sequences)

SEQ ID NO: 1 - protein, 8 aa, Artificial Sequence
  Synthetic peptide
EVNLAAEF

SEQ ID NO: 2 - protein, 15 aa, Artificial Sequence
  Synthetic peptide
RKTEEISEVNLDAEF

SEQ ID NO: 3 - protein, 15 aa, Artificial Sequence
  Synthetic peptide
RKTEEIXEVNLDAEF

SEQ ID NO: 4 - protein, 15 aa, Artificial Sequence
  Synthetic peptide
RKTEEXSEVNLDAEF

SEQ ID NO: 5 - protein, 15 aa, Artificial Sequence
  Synthetic peptide
RKTEXISEVNLDAEF

SEQ ID NO: 6 - protein, 15 aa, Artificial Sequence
  Synthetic peptide
RKTXEISEVNLDAEF

SEQ ID NO: 7 - protein, 14 aa, Artificial Sequence
  Synthetic peptide
RTEEXSEVNLAAEF

SEQ ID NO: 8 - protein, 14 aa, Artificial Sequence
  Synthetic peptide
RTEXISEVNLAAEF

SEQ ID NO: 9 - protein, 14 aa, Artificial Sequence
  Synthetic peptide
RTXEISEVNLAAEF

SEQ ID NO: 10 - protein, 14 aa, Artificial Sequence
  Synthetic peptide
RTEEISEVNLDAEF

SEQ ID NO: 11 - protein, 15 aa, Artificial Sequence
  Synthetic peptide
GSAKVAFSAIRSTNH

SEQ ID NO: 12 - protein, 7 aa, Artificial Sequence
  Synthetic peptide
GSAKVAF

SEQ ID NO: 13 - protein, 8 aa, Artificial Sequence
  Synthetic peptide
SAIRSTNH

SEQ ID NO: 14 - protein, 15 aa, Artificial Sequence
  Synthetic peptide
GSAKVAFSAIRSTNH

SEQ ID NO: 15 - protein, 46 aa, Artificial Sequence
  Synthetic peptide
KVDENMVIDETLDVKEMIFNAERVGGLEEERESVGPLREDFSLSSS

SEQ ID NO: 16 - protein, 46 aa, Artificial Sequence
  Synthetic peptide
PEKEKMNPLEQYERKVNASVPRGFPFHSSEIQRDELAPAGTGVSRE

SEQ ID NO: 17 - protein, 20 aa, Artificial Sequence
  Synthetic peptide
HLRAQDLHAERSAAQLSLSS

SEQ ID NO: 18 - protein, 14 aa, Artificial Sequence
  Synthetic peptide
SDYEALTLQAKEFQ